(19) United States
(12) Patent Application Publication
(10) Pub. No.: US 2023/0201832 A1
(43) Pub. Date: Jun. 29, 2023

(54) A method and device for automated and point-of-care nucleic acid amplification test

(71) Applicant: The Board of Trustees of the Leland Stanford Junior University, Stanford, CA (US)

(72) Inventors: Chengyang Yao, Palo Alto, CA (US); Katie A. Antilla, Stanford, CA (US); Ana Sofia de Olazarra, Menlo Park, CA (US); Neeraja Ravi, Stanford, CA (US); Elaine Ng, Redwood City, CA (US); Shan X Wang, Portola Valley, CA (US)

(21) Appl. No.: 17/928,581

(22) PCT Filed: Jun. 30, 2021
(86) PCT No.: PCT/US2021/039883

§ 371 Date: Nov. 29, 2022

Related U.S. Application Data
(60) Provisional application No. 63/046,265, filed on Jun. 30, 2020.

(51) Int. Cl. B01L 3/00; B01L 7/00; C12Q 1/6844

(52) U.S. Cl. CPC B01L 3/502761; B01L 7/52; C12Q 1/6844; B01L 2200/0668; B01L 2200/16; B01L 2300/0663; B01L 2300/0645; B01L 2300/1827; B01L 2200/0621

(57) ABSTRACT

This work provides a method and device for performing quantitative and sensitive multiplex nucleic acid detection at the point-of-care using magnetoresistive (MR) detection. Temperature calibration of the MR sensor elements is performed per-element, rather than assuming the same calibration parameters apply to each element of the MR sensor array. It can include a digitally controlled fluidic system to allow automated wash and reagent injection, an on-chip temperature management system to achieve on-chip polymerase chain reactions (PCR), and a portable magnetoresistive sensor platform. This approach requires minimal user involvement beyond adding the sample and simple top-level control, making it highly desirable for point-of-care applications.

### FIELD OF THE INVENTION

This invention relates to nucleic acid amplification and magnetic biosensing devices and techniques.

### BACKGROUND

Biological assays based on the polymerase chain reaction (PCR) are important tools for detecting biological sample with high specificity and sensitivity. Detection of bound samples in PCR is typically done with fluorescent detection. However, magnetic detection for PCR assays has also been considered in the art. Magnetic detection in PCR relies on magnetoresistive (MR) sensors whose resistance depends on the magnetic field. Thus a bound sample that is also bound to a magnetic nanoparticle can be detected magnetically because of the effect of the magnetic nanoparticle on the nearby magnetic field. However, the resistance of MR sensors also depends on their temperature. Accordingly, accurate control and measurement of temperature is important in MR detection for PCR assays.

An approach for temperature management in MR detection considered in US 2018/0313789 is to determine parameters R₀ and α in the calibration equation R=R₀(1+α(T−T0)) for zero-field resistivity. Once this is done, the MR sensors themselves can be used as temperature sensors. An important feature of this work is that it relates to an array of MR sensors, and it is implicitly assumed that the same α and R₀ can be used for every sensor in the array.

We have found that this assumption may not be justified in practice. Accordingly, it would be an advance in the art to provide improved temperature management for MR detection in PCR assays.

### SUMMARY

In this work, a per-element temperature calibration of the MR sensors is performed so that they can more accurately provide temperature measurements for controlling PCR cycling.

One exemplary embodiment is a device for portable on-chip nucleic acid amplification and endpoint or real-time detection via PCR and magnetic biosensing, respectively. Feature of this preferred embodiment include the following, individually or in any combination.

1) A Joule heating network including surface-mount resistors for implementation of controlled heating during thermal cycling. A schematic of this resistor network, which is physically located on the bottom layer of the printed circuit board (PCB), is pictured in FIGS. 7A-C. As FIG. 8 indicates, the heat generated by the resistors is conveyed to a metalized heating plate (located on the top layer) through a plugged via. This configuration enables more efficient vertical transfer, and uniform distribution, of heat to the region containing the PCR solution.
2) A temperature-sensing mechanism that relies upon measured variations in the resistance of the magnetic sensors that are caused by changes in temperature.
3) A magnetic sensor array that performs endpoint detection or real time detection of the analyte of interest.
4) A custom circuitry system that has an analog front end to drive and read from the magnetic sensor array, an analog-to-digital converter module to digitize the sensor data, and a micro-controller to control different modules and perform frequency analysis of the acquired information.
5) A mobile phone application to enable user specification of both thermal cycling and endpoint read-out parameters. For the thermal cycling component, users can specify: the temperature and length of time associated with each step, as well as the number of cycles associated with the amplification process. For the read-out component, users are able to specify: the incubation time and the read-out time. Upon completion of the assay, the user is presented with their personal assay results and the opportunity to forward those results to their medical care provider.

An exemplary end-point PCR approach with this apparatus includes amplification of DNA on a chip, followed by quantification of the resulting PCR product using magnetic sensing. Significant features of preferred end-point assays include the following, individually or in any combination.

1) The device allowing for both temperature cycling and magnetoresistive signal reading on the same chip.
2) A DNA oligonucleotide primer (either the forward or the reverse primer) functionalized with biotin, to allow for eventual binding to the streptavidin on the surface of the magnetic nanoparticles.
3) An additional two temperature steps added after the PCR temperature cycling finishes. These two additional steps are: a final denaturation step to re-separate all the newly formed DNA strands, then a “detection” step at a lower temperature during which the biotinylated target DNA hybridizes with the surface capture probe and the streptavidin-coated magnetic nanoparticle binds to the biotin, tethering the nanoparticle to the surface (see (E) of FIG. 1).
4) A PCR supermix with low glycerol content, so that the viscosity of the solution doesn't disrupt/slow nanoparticle diffusion too much.

An exemplary real-time PCR approach with this apparatus includes amplification of DNA on a chip, followed by quantification of the resulting PCR product using magnetic sensing. Significant features of preferred real-time assays include the following, individually or in any combination.

1) The device allowing for both temperature cycling and magnetoresistive signal reading on the same chip.
2) A DNA polymerase enzyme that has antibody-mediated rather than chemically-mediated HotStart properties, so that once it is activated at the beginning of the PCR, it will not be deactivated by the lower temperature involved in the detection step.
3) A secondary detection probe (a single-stranded DNA oligo functionalized with biotin). The secondary detection probe can be designed with a sequence independent of the sequences of any of the target genes, so that the same probe can be used to detect multiple targets.
4) An additional two temperature steps added to the standard PCR temperature cycle (denaturation, annealing, extension). These two additional steps are: a second denaturation step to re-separate all the newly formed DNA strands, then a “detection” step at a lower temperature during which the target DNA hybridizes with the surface capture probe and the secondary detection probe, forms a sandwich structure tethering a magnetic nanoparticle to the surface (see (E) of FIG. 2).
5) A PCR supermix with low glycerol content, so that the viscosity of the solution doesn't disrupt/slow nanoparticle diffusion too much.

### BRIEF DESCRIPTION OF THE DRAWINGS

FIG. 1 shows a preferred end-point PCR assay for use in embodiments of the invention.

FIG. 2 shows a preferred real-time PCR assay for use in embodiments of the invention.

FIG. 3 shows an exemplary block diagram relating to embodiments of the invention.

FIG. 4 is a flow chart showing an exemplary PCR process sequence.

FIG. 5 shows exemplary temperature calibration data.

FIG. 6 is an exemplary histogram of MR sensor resistance.

FIGS. 7A-C show several views of an exemplary printed circuit board configuration including heating resistors and the MR sensor chip.

FIG. 8 shows an exemplary configuration for the PCR reaction chamber.

FIG. 9 is a circuit schematic relating to analog filtering for double modulation.

FIG. 10 is a circuit schematic relating to temperature control for PCR cycling and control of the pumps.

FIG. 11 is a circuit schematic relating to driving the magnetic coil to provide the biasing magnetic field.

FIG. 12 shows an exemplary configuration for the reaction well and pump channels.

FIG. 13 shows exemplary PCR temperature cycling data.

FIG. 14 is a measured binding curve for end-point PCR.

FIG. 15 shows multiplex assay results for end-point PCR.

FIG. 16 shows an example of active cooling for PCR temperature cycling.

### DETAILED DESCRIPTION

To better appreciate the context of the present approach, it is helpful to first consider two preferred PCR assay approaches.

### A) End-Point PCR

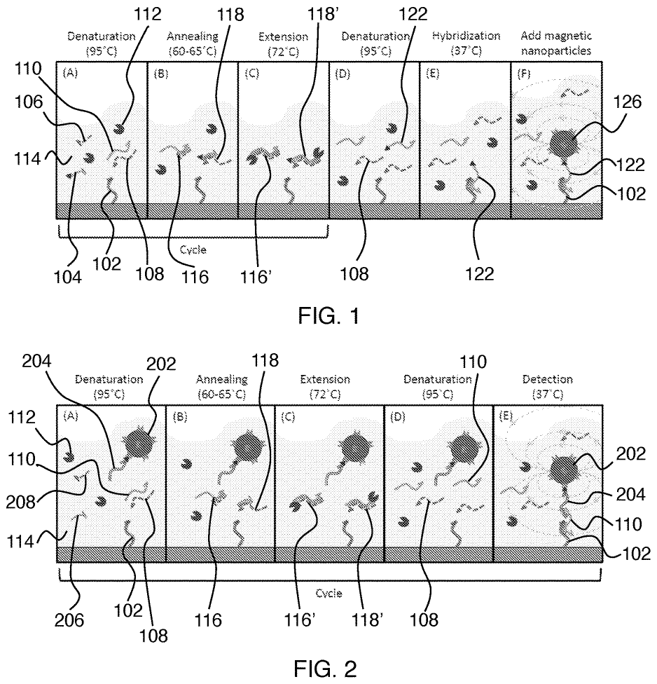

The steps of a preferred method for on-chip PCR with end-point detection are illustrated in FIG. 1. For simplicity, only one sensor and one set of target DNA/primers/capture probes are shown, and elements that are the same from one step to the next are mostly not referenced in each step.

Step (A) shows a PCR reaction mixture 114 including DNA polymerase enzymes 112, double-stranded template DNA (sense strand 110 denoted by a solid line, antisense strand 108 denoted by dashed line), dNTPs (not shown for simplicity), and forward and reverse oligonucleotide primers (104 and 106 respectively), the forward primer 104 being functionalized with biotin (triangle). The PCR reaction mixture is disposed in a reaction well on the MR sensing chip as described below. The MR sensing chip is functionalized with oligonucleotide surface capture probes 102 that are complementary to the target sequence prior to the addition of the PCR reaction mixture to enable subsequent binding (see U.S. Pat. No. 7,906,345, hereby incorporated by reference in its entirety, for details on using nanotags as detection probes). The liquid in the well is then heated (e.g., to around 95° C.) to denature the double-stranded template DNA.

Step (B) show the result of cooling the reaction mixture (actively or passively) to a specified annealing temperature—typically in the range of 60-65° C. —to enable the primers to anneal to the template DNA strands as shown. The resulting double-stranded intermediate reaction products are referenced as 116 and 118.

Step (C) shows the result of heating the liquid in the well to an intermediate elongation temperature—typically around 72° C., or the temperature at which the DNA polymerase is most active—allowing it to create copies of the template DNA strands. This elongation causes 116 to become 116′ and 118 to become 118′. This sequence of steps (A)-(C) is repeated for a user-specified number of cycles (generally within the range of 20-40 cycles). The result of this cycling is to greatly increase the concentration of the target DNA having sense strand 110 and anti-sense strand 108 in the reaction mixture.

Upon completion of the thermal cycling, the liquid in the well is heated to approximately 95° C. one final time in order to denature all of the double-stranded PCR products, as shown in step (D). Here 122 shows biotinylated sense strands.

Step (E) shows the result of cooling the liquid in the well to a much lower temperature (around 37° C.) to allow the biotinylated sense strands 122 to hybridize to the surface capture probes 102.

Step (F) shows the result of adding a solution of magnetic nanoparticles to the well; the streptavidin on the surface of the nanoparticle 126 binds to the biotin on the 5′ end of the DNA sense strand 122, tethering the nanoparticle to the surface and allowing the magnetoresistive sensor beneath to detect a signal. The magnetoresistive signal measured is directly proportional to the concentration of the target DNA sequence in the sample.

### B) Real-Time PCR

The assay schematic of a preferred method for on-chip real-time PCR is shown in FIG. 2. Similar to FIG. 1, only one sensor and one set of target DNA/primers/capture probe are shown.

Step (A) shows a PCR reaction mixture 114 including DNA polymerase enzymes 112, double-stranded template DNA (sense strand 110 denoted by solid line, antisense strand 108 denoted by dashed line), dNTPs (not shown for simplicity), and forward and reverse oligonucleotide primers (206 and 208 respectively). The reaction mixture also includes a 5′ biotinylated secondary detection probe 204 (double line), which is attached to a magnetic nanoparticle 202 via biotin (triangle)-streptavidin (concave pentagon) binding. Oligonucleotide surface capture probes 102 complementary to the sense strand are attached to the surface of the MR sensor chip (see U.S. Pat. No. 7,906,345, hereby incorporated by reference in its entirety for details on using nanotags as detection probes). The liquid in the well is then heated up to a high temperature (around 95° C.) to denature the double-stranded template DNA.

Step (B) shows the result of cooling the liquid in the well down to a specific temperature, generally in the range of 60-65° C., to allow the primers to anneal to the template DNA strands. The resulting double-stranded intermediate reaction products are referenced as 116 and 118.

Step (C) shows the result of heating the liquid in the well to an intermediate temperature (around 72° C.) at which the DNA polymerase has highest activity, allowing it to create copies of the template strands. This elongation causes 116 to become 116′ and 118 to become 118′.

Step (D) shows the result of heating the liquid in the well up to high temperature (around 95° C.) to denature the newly-formed double-stranded DNA.

Step (E) shows the result of cooling the liquid in the well to a much lower temperature (around 37° C.) to allow the sense template DNA strand 110 to hybridize with the surface capture probe 102 and to the secondary detection probe 204, forming a sandwich structure that tethers the nanoparticle 202 to the surface and allows the GMR sensor beneath to detect a signal. The GMR signal measured is directly proportional to the concentration of the target DNA sequence in the sample. This sequence of steps (A)-(E) is repeated for the desired number of cycles (generally within the range of 20-40 cycles), with the quantity of DNA present at the end of each cycle being measured by the GMR sensor during step (E).

Two significant features of preferred embodiments that improve quantification of target DNA on-chip are as follows. First, the magnetic tags that we use are colloidally stable in solution. Therefore, incubating a solution of tags over our sensors does not induce any signal in our sensors until a detection target functionalized with biotin is present and bound to a captured antigen. Second, in the on-chip real-time PCR assay, the secondary detection probe 204 is biotinylated (rather than directly biotinylating one of the primers as in the endpoint PCR assay of FIG. 1) because once the streptavidin-coated nanoparticles are added at the beginning of the assay, they will quickly bind to the biotin, and if they are bound to either of the primers, they will slow down primer diffusion too much, making it more difficult for the primers to anneal to the target DNA and thus decreasing the PCR amplification efficiency.

In the examples of FIGS. 1-2, a polymerase is used that has distinct annealing and extension temperatures. It is also possible to use a polymerase that performs annealing and extension at the same temperature, in which case the separate annealing and extension steps described above consolidate into a single step.

A detailed example of a real-time PCR assay follows.

1) Functionalize a capture probe (a single-stranded DNA oligo) onto the chip surface directly over each GMR sensor. A covalent chemistry can be used utilizing primary amino groups attached to the 5′ ends of the DNA.
2) Block the surface of the sensor to prevent non-specific binding. In order to block the surface, a blocking buffer (for example, 1% BSA (bovine serum albumin) in PBS (phosphate-buffered saline)) can be added to the reaction well for one hour.
3) Remove the blocking buffer and wash the surface of the chip with a wash buffer (for example, 0.1% BSA and 0.05% Tween-20 in PBS) and/or with water, then air dry the chip (Note that steps 1-3 of the assay can be done prior to delivery of the chip, so that no wash steps are required for the end user.)
4) Prepare desired PCR reaction mixture containing DNA polymerase, dNTPs, salts/buffers, primers, sample DNA, and secondary detection probe (a single-stranded DNA oligo that is functionalized with biotin, or an alternate chemistry), then transfer to reaction well and plug cartridge into GMR reader station.
5) Add in a solution of magnetic nanoparticles that are functionalized with streptavidin (or an alternate chemistry). In our platform, a total reaction volume (PCR mix plus nanoparticle solution) of 100-200 μL is used.
6) Input desired temperature cycling parameters (temperatures, time spent at each temperature) and begin on-chip temperature cycling. The steps in each temperature cycle include (in order):
6a) Denaturation (for example, 95° C. for 30 seconds), during which all double-stranded DNA strands separate.
6b) Annealing (for example, 60-65° C. for 30 seconds), during which the forward and reverse primers hybridize with the template DNA strands.
6c) Extension (for example, 72° C. for 10 seconds), during which the DNA polymerase replicates each template DNA strand with an attached primer.
6d) Denaturation (for example, 95° C. for 30 seconds), during which all double-stranded DNA again separates into single strands.
6e) Detection (for example, 37° C. for 2 minutes), during which both a secondary detection probe and a surface capture probe hybridizes to each template DNA strand, forming a sandwich structure. The biotin on the secondary detection probe will be bound to the streptavidin on the surface of a magnetic nanoparticle, so this sandwich structure will tether the nanoparticle to the surface of the GMR sensor, causing a signal to be detected.
7) The GMR signals measured at the end of the detection step in each cycle can be plotted together vs. cycle number, giving a quantitative graph of DNA amplification over the course of the run.

### C) Hardware Components

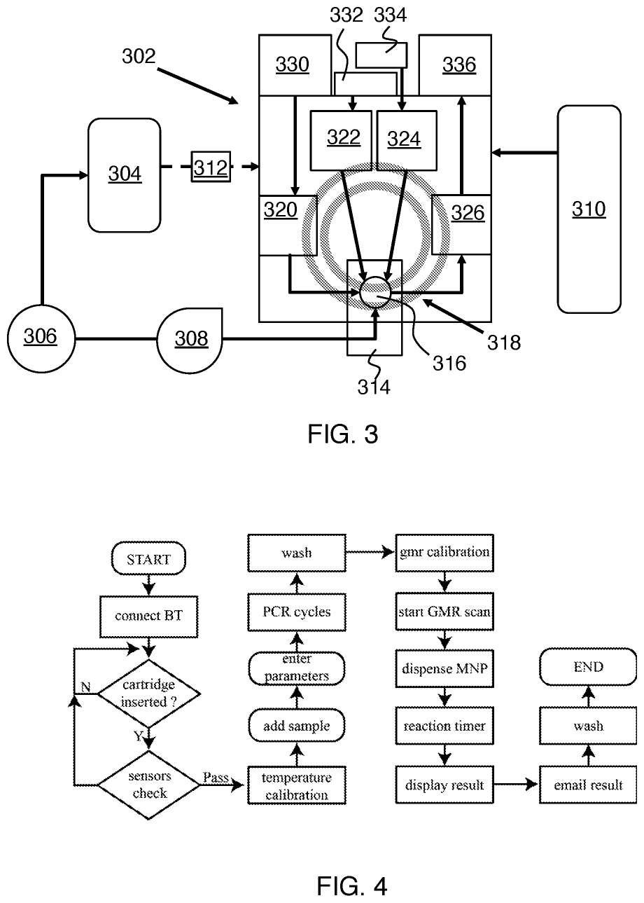

A high-level schematic block diagram of a preferred embodiment is included in FIG. 3. The major components are a power supply 310 (e.g., including a battery and/or an AC adapter), a cartridge 314 including a reaction well 316 that also houses a chip carrier printed circuit board, a magnetic coil 318 wrapped around a toroid iron powder core, a custom printed circuit board housing the data acquisition circuits, four pumps (320, 322, 324, 326), four fluid reservoirs (330, 332, 334, 336), and a smartphone 304 for top level user control. Here reservoirs 330, 332, 334, 336 are for a wash solution, reagents, magnetic nanoparticles and waste fluid, respectively. FIG. 3 also shows their relative positions inside a case 302. The device can be powered by either a portable battery, or a power adapter that directly plugs into a wall AC outlet. In operation a user 306 provides a sample 308 to the device and controls the cycling with top-level controller 304 (e.g., a smartphone). Optionally, an intermediate controller 312 can be used to provide lower level control of the system and an easier interface to top-level controller 304.

To use this device to perform an assay, the user manually inserts the cartridge into the connector surrounded by the coil. The coil and its core have an open cutout in the place of the cartridge. The user then adds the sample into the open reaction well that is located on top of the cartridge and presses a “start” button on the smartphone. The smartphone then communicates with the micro-controller in the device to complete the assay according to the predetermined steps. An information architecture of this program is illustrated in FIG. 4.

### D) Temperature Management

A major feature of this work is precise management of temperature. This is achieved by a feedback proportional-integral-differential control system. The temperature of the reaction mix is first sensed by utilizing the magnetoresistive sensors. Because the sensors are located on top of the silicon chips, they are only separated from the reaction liquid by a thin layer of silicon dioxide. This enables the variations in the resistances of the sensors to reflect variations in the temperature of the reaction liquid, hence allowing the sensors to be used as an effective temperature-sensing mechanism. To increase the temperature of the reaction mix, we utilized joule heating by passing current into four resistors underneath the sensor chip. Because the required temperatures for PCR are almost always higher than room temperature, there is always a passive cooling effect with the surrounding environment. By controlling the heating power, the reaction mix temperature can both increase and decrease within the range of 25° C. to 100° C.

The temperature dependence of the resistance of the magnetoresistive spin valve sensors has been reported in the literature; however, it is typically presented as a non-ideality responsible for inducing signals that are indistinguishable from the signals associated with the magnetic nano-tags. In this work, we utilize the same temperature-resistance relationship to detect the temperature change of the sensor as a proxy for the temperature of the reaction fluid. The temperature dependence of a resistor can be written as shown in Eq. 1, where R₀ is the resistance at a reference temperature T₀, and α is the temperature coefficient.

R=R₀(1+α(T−T₀)) (1)

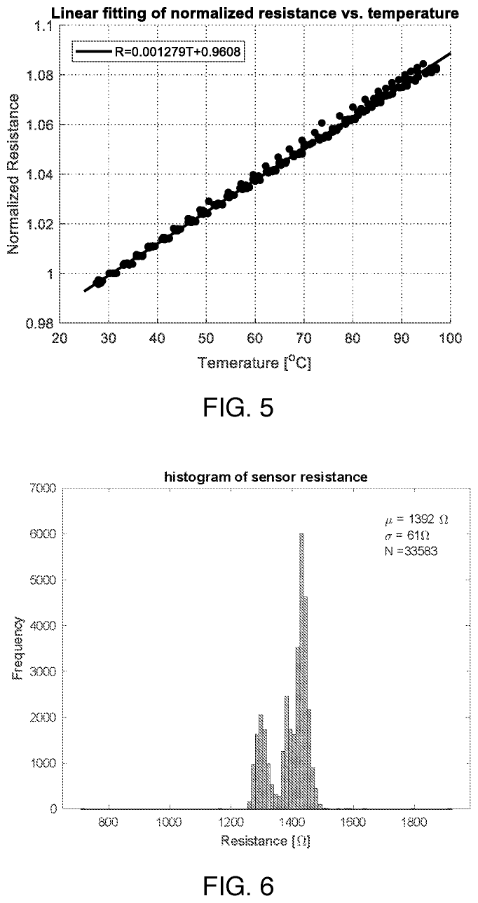

In order to determine the temperature coefficient for a magnetoresistive sensor, a series of experiments were carried out in which the sensors were heated to 100° C., then allowed to cool while the temperatures and resistances of the system were recorded. FIG. 5 plots the measured results along with a linear fit, the extracted temperature coefficient α is 1279 ppm/° C. It is important to note that this coefficient strongly depends on the materials used to manufacture the sensor and subsequent device fabrication steps.

To accurately detect the temperature, a calibration step is required at the beginning of each assay. This is because process variations can cause changes to the nominal resistance from sensor to sensor, even on the same chip. FIG. 6 shows a histogram of 33583 sensor resistance from 420 chips that are taken from two wafers. Inter-die and inter-wafer process variation are clearly reflected by the two peaks in the distribution.

Because of this resistance variation, it is impossible to have a single value for R₀ for all the sensors. Instead, the reference R₀ⱼ needs to be recorded for every sensor before each assay as a calibration step. A thermistor is placed near the magnetoresistive chip on the same carrier PCB to acquire the reference temperature T₀ before the assay and also to establish the reference point. Accordingly, the temperature calibration provides values for R₀ⱼ for each sensor j in the array, resulting in the temperature calibration of Eq. 2.

R=R₀ⱼ(1+α(T−T₀)) (2)

The coefficient α is taken to be the same for every sensor in the array. The reference temperature T₀ is also taken to be the same for every sensor in the array.

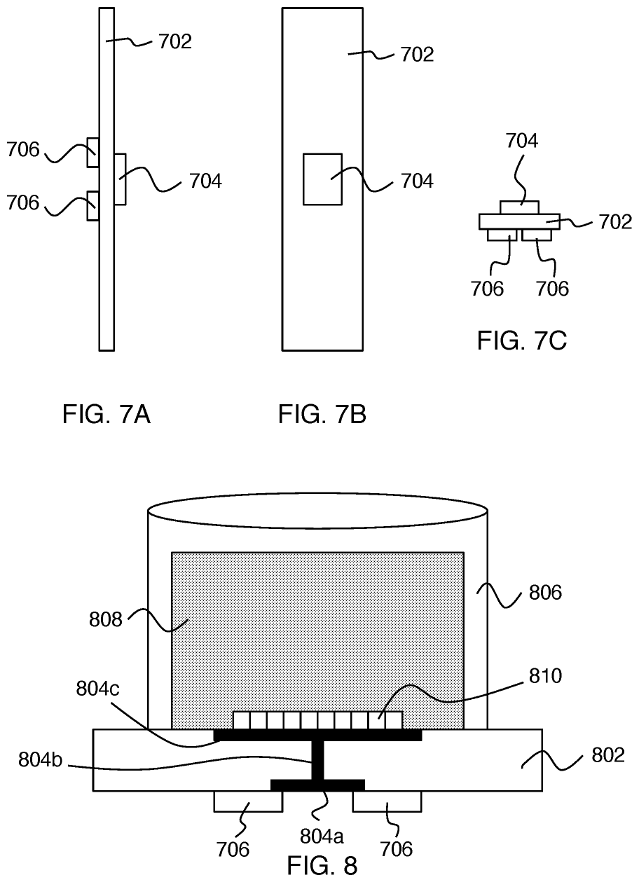

A drawing of the magnetoresistive chip carrier PCB is shown in FIGS. 7A-C. FIG. 7A is a side view, FIG. 7B is a top view, and FIG. 7C is an end view. PCB 702 houses the thermistor and heating resistors 706, and it also connects the magnetoresistive chip 704 to the reader station.

A cross-section view of the heating resistors and magnetoresistive sensor is shown in FIG. 8. Here 802 is the printed circuit board, 806 is the reaction well, 808 is the reaction fluid, and 810 is the MR sensor chip. The example of FIG. 8 also includes thermally conductive plates 804a and 804c connected by thermally conductive via 804b. Such thermally conductive plates and vias can be made of metallic traces and the like as is well known in PCB technology. Preferred embodiments include features 804a-c to improve heat flow from heating resistors 706 to MR chip 810 and reaction fluid 808.

### E) Signal Processing Circuits

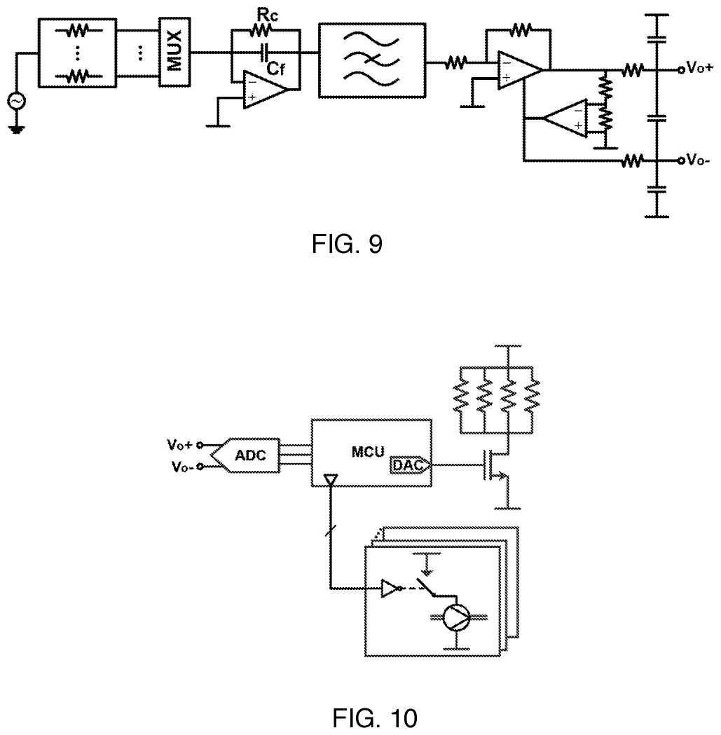

In preferred embodiments, the device uses a series of analog filters to acquire a double modulated signal from the magnetoresistive sensor arrays. The double modulation technique is described in U.S. Pat. No. 8,405,385, hereby incorporated by reference in its entirety. Simply put, the double modulation scheme separates the resistive and magnetoresistive components of the sensor by modulating them to different frequencies. A major challenge when applying double modulation to magnetoresistive sensors is that the resistive component tends to be approximately 40 dB higher than the magnetoresistive component as seen on the spectrum. Instead of using a duplicated path to suppress the non-magnetic tone, as previously reported, we use a series of analog filters on the signal path, as shown in FIG. 9. This new signal conditioning path can successfully suppress the magnitude of the resistive component in the frequency domain while reducing the offset and noise introduced by the additional signal path and component mismatch.

Nucleic acid amplification tests often require the temperature to increase beyond 90° C., which corresponds to a resistance increase of over 8%, as seen in FIG. 5. When using the duplicated path method to suppress the resistive component, the resistive component from the sensor will increase due to the increasing temperature, but the duplicate path resistance remains the same as the reference resistors are not being heated. This can make the use of a duplicate path problematic because it can lead to inaccurate temperature tracking. In particular, if the sensor resistance is slightly lower than the reference resistance at room temperature, but then increases to a value higher than the reference resistance, the resistive component in the frequency domain will be made to decrease to zero, then increase due to the subtraction, making temperature tracking inaccurate. Ideally the canceling resistor should be slightly lower in resistance than the magnetoresistive sensors, but this would require a prohibitively large resistor array, especially for a point-of-care device. By using a series of analog band shaping filters, the resistive tone always sees constant gains regardless of the temperature. Therefore, the system is able to accurately track the sensor temperature.

In this work, we utilize the same signal path for magnetic resistance to read the sensors' nominal resistance and infer the temperature from this temperature/resistance relationship. The feedback loop is depicted in FIG. 10. When reading the temperature information, the magnetic field is turned off to save power and reduce noise, since only the nominal resistance is acquired. After acquiring the sensor resistance, the temperature is calculated according to Eq. 2. The micro-controller then computes a voltage output value for the NMOS' gate voltage. The NMOS controls how much power is dissipated through the heating resistors. A proportional-integral-differential control algorithm was developed and tuned for this application.

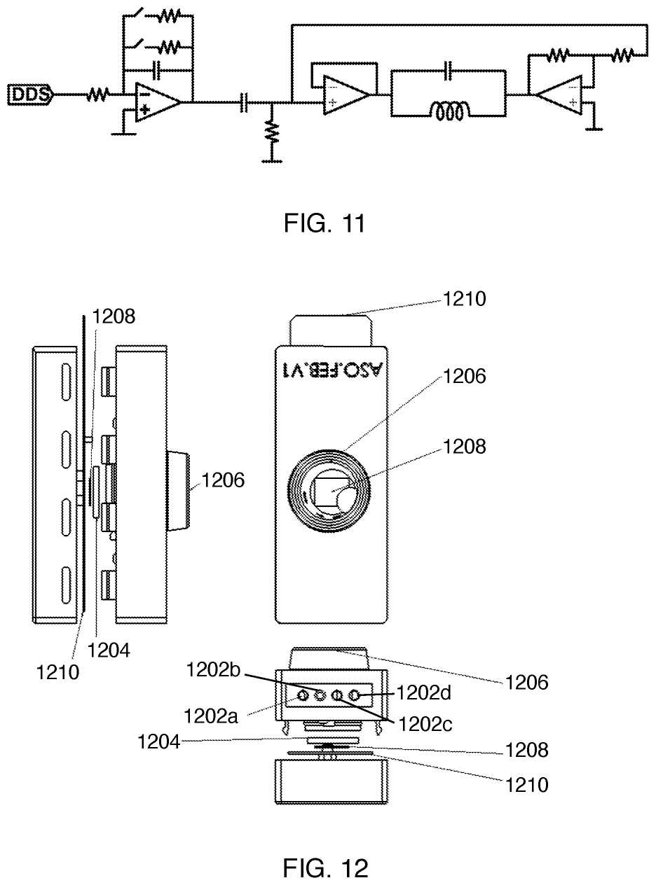

The micro-controller also has dedicated output pins to control the pumps by turning on and off a digital switch that connects and disconnects the pumps to the power line, as illustrated in FIG. 10. The circuit used to drive the coil and generate magnetic field is illustrated in FIG. 11. The coil is modeled as an inductor here. To maximize the power efficiency, a capacitor is connected in parallel with the coil to match its impedance. There is a digitally controlled switch to select the gain resistor of the operational amplifier. This switches the magnetic field between 2 mT and 3 mT, for the purpose of magnetic sensitivity calibration.

### F) Fluidic System Design

Traditionally a trained technician is required to perform a nucleic acid amplification test or a protein test, necessitating the use of central laboratory facilities. This work overcomes many central laboratory limitations by featuring an automated detection system in which the user is only required to manually add the sample to the cartridge, then press a button on a smartphone application in order to initialize the assay. The fluidic system has four pumps, each connects the reaction well to a liquid chamber, as depicted in FIG. 3. The cartridge has two pieces, and a reaction well is created by sandwiching the chip carrier PCB between the top and bottom piece. There is a hollow well in the top piece surrounding the magnetoresistive sensor chip, as illustrated in FIGS. 8 and 12. To seal the gap between the top cartridge and the PCB, an O-ring 1204 is placed around the well. Other features on FIG. 12 are as follows. Here 1202a, 1202b, 1202c, and 1202d are the piping for reagents (and correspond to pumps 320, 322, 324, and 326 on FIG. 3, respectively), 1206 is the opening for the reaction well, 1208 is the MR sensor chip and 1210 is the PCB.

As illustrated in FIG. 3, there are four pipes in the cartridge to guide the reagents and wash buffer into and out of the reaction well. Three of the pipe outlets are located on the top of the well and one is at the bottom of the well. The three on top are used to inject reagents into the well and the one on bottom is designed to aspirate from the well into a waste buffer. Pumps 320, 322, 324 are unidirectional, allowing reagents to only flow into—not out of—the well in order to prevent contamination. However, pump 326 is set up to be bidirectional so that it can pump a small amount of liquid in and out to agitate the mix in the reaction well. This can facilitate binding kinetics of the reagents.

### G) Experimental Example

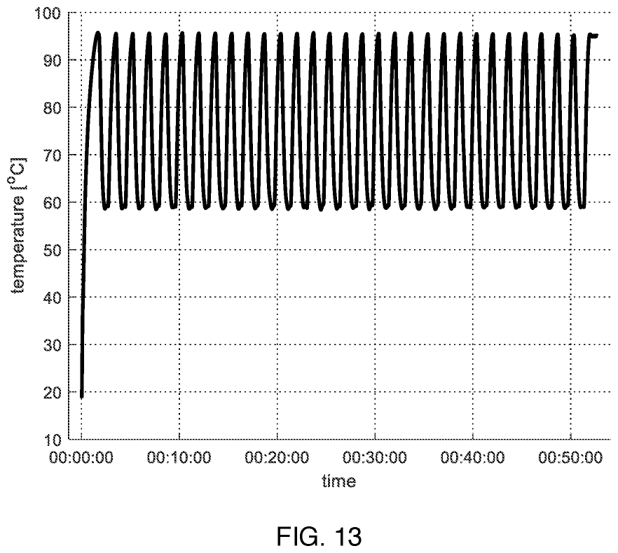

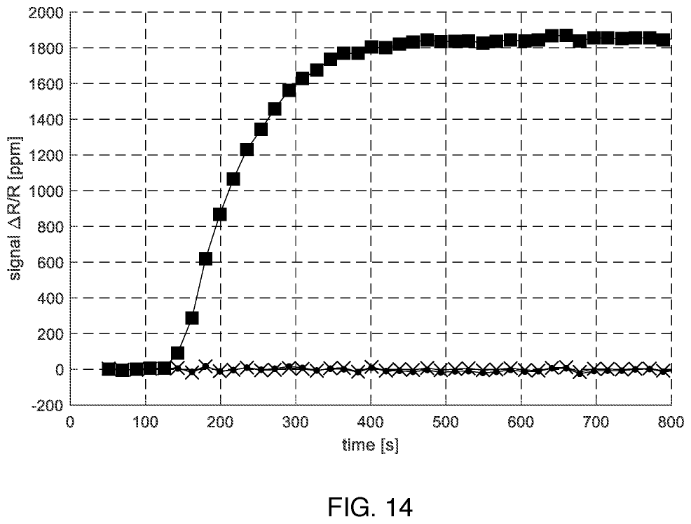

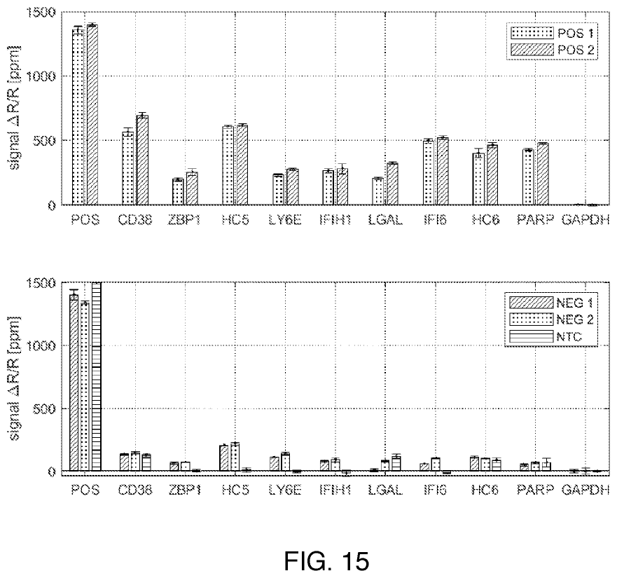

Here we consider a multiplexed genetic signature measurement as an example of application. FIG. 13 shows the temperature curve for a PCR protocol of 30 thermal cycles. An end-point quantitative measurement of the DNA copies is carried out by the giant-magnetoresistive sensors and double modulation readout method. FIG. 14 shows binding curves of the captured DNA and the detection magnetic nanoparticles as described in step (F) of FIG. 1. Here the “▪” curves correspond to sensors immobilized with the correct capture probes and analyte DNA while the “●” and “X” curves with very little signal correspond to empty sensors and sensors spotted with zero analyte probes. The zero of the time axis is the start of step (F) of FIG. 1, and the magnetic nanoparticles are added 120 seconds after that. The end-point measurement result of a multiplexed gene expression assay is shown in FIG. 15, showing consistent and excellent separation of positive, negative, and no-template samples.

### H) Variations

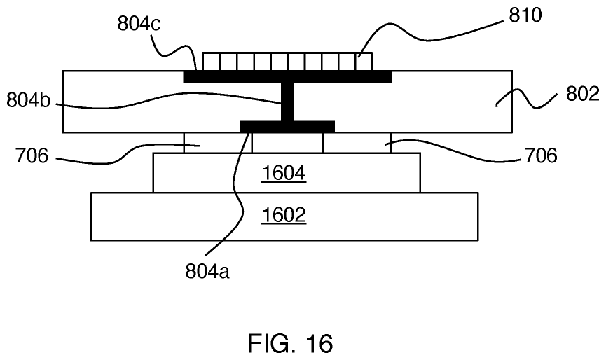

The preceding examples have made use of passive cooling for the PCR cycling, which is often preferred to simplify the overall design. However, in some cases active cooling may be preferred. FIG. 16 shows an example of such a configuration. Here 1602 is a heat sink and 1604 is a thermoelectric element (e.g., a Peltier cooler), and the remaining elements are as referenced in connection with FIG. 8.

Another variation is to include a “zone control” in the reaction temperature. Because we have per element temperature sensing, we can construct a temperature map using the MR sensor array. If multiple heating resistors are used, they can be individually controlled to provide improved local control of the heat distribution. Since each heating resistor is most effective in heating the regions immediately above it, we are able to adjust the local temperature there. This can further ensure there is no temperature gradient in the reaction mix. In other words, two or more heating elements can be individually controlled in response to temperature signals from the MR sensor elements to reduce temperature variation of the MR sensor elements.

### I) Exemplary Embodiments

An embodiment of the invention is an apparatus for performing point of care PCR (polymerase chain reaction) testing, where the apparatus includes:

a reaction chamber (e.g., 806 on FIG. 8) configured to hold PCR reagents;

a magnetoresistive sensor array (e.g., 810 on FIG. 8) disposed within the reaction chamber where the magnetoresistive sensor array includes two or more sensor elements (schematically shown on FIG. 8);

one or more heating elements (e.g., 706 on FIG. 8) disposed to provide heat to the reaction chamber;

a processor (e.g., 304 and/or 312 on FIG. 3) configured to provide temperature control of the reaction chamber for PCR cycling by controlling the one or more heating elements responsive to signals from the temperature sensors.

The two or more sensor elements are used as temperature sensors for the reaction chamber. In this embodiment, temperature calibration is performed as follows. Let T₀ be a reference temperature, let R₀ⱼ be zero field resistivity of sensor element j at temperature T₀, let α be a temperature coefficient of the magnetoresistive sensor array, and assume a zero field resistivity Rⱼ of sensor element j is given by Rⱼ=R₀ⱼ(1+α(T−T₀)). Temperature calibration of the sensor elements is determined by determining α for the magnetoresistive sensor array and determining R₀ⱼ for each sensor element.

The apparatus of the preceding example can further include a substrate (e.g., 802 on FIG. 8). Here the magnetoresistive sensor array (e.g., 810 on FIG. 8) is disposed on a first surface of the substrate. The one or more heating elements (e.g., 706 on FIG. 8) are disposed on a second surface of the substrate opposite the first surface of the substrate, as shown. A floor of the PCR reaction chamber is formed by the substrate such that the magnetoresistive sensor array is inside the PCR reaction chamber, as shown.

Optionally this embodiment can further include one or more thermally conductive vias (e.g., 804b on FIG. 8) configured to enhance heat flow from the one or more heating elements to the PCR reaction chamber. It can also further include a thermally conductive plate (e.g., 804c on FIG. 8) sandwiched between the magnetoresistive sensor array and the substrate, where the thermally conductive plate is in physical contact with the one or more thermally conductive vias.

Temperature in the PCR reaction chamber can be measured by averaging the temperatures obtained from each sensor element. The temperature coefficient of the magnetoresistive sensor array a can be determined in a separate calibration step. The reference temperature T₀ can room temperature, and determining R₀ⱼ for each sensor element j can be done by measuring zero-field resistivity at room temperature for each sensor element.

The processor can be configured to provide end-point PCR detection (e.g., as on FIG. 1) or the processor can be configured to provide real-time PCR detection (e.g., as on FIG. 2). The PCR reagents can include a secondary detection probe (e.g., 204 on FIG. 2) configured to bind to magnetic nanoparticles and configured to bind to a target species to be detected.

The real-time PCR detection can cycle between a detection phase (e.g., (E) on FIG. 2) and non-detection phases (e.g., (A) through (D) on FIG. 2), where the secondary detection probe is bound to the target species during the detection phase, and where the secondary detection probe is not bound to the target species during the non-detection phases.

The secondary detection probe can be complementary to a common sequence included in the PCR primers for every target species, whereby the secondary detection probe can be used universally for all target species in a multiplexed real-time PCR reaction.

The current provided to the sensor elements can be modulated at a first frequency f₁, and a magnetic field provided to the sensor elements can be modulated at a second frequency f₂ distinct from the first frequency. The resulting signal of interest from the sensor elements is at a sum frequency f₁+f₂ or at a difference frequency f₁−f₂. This is an example of double modulation as referred to above. If double modulation is used, analog filtering circuitry configured to pass the signal of interest while suppressing other signals at frequencies f₁ and f₂ can be used.

In preferred embodiments, the PCR reagents include a DNA polymerase having an antibody-mediated hot start property, whereby the DNA polymerase will not deactivate during lower-temperature hybridization steps (e.g., having a temperature of roughly 37 C) of the PCR cycling.

The PCR reagents preferably have a low glycerol content (0.5% or less glycerol, more preferably 0.25% or less glycerol, by volume), whereby an effect of fluid viscosity on magnetic nanoparticle diffusion is negligible.

The heating elements can be resistive Joule heating elements. Cooling for the PCR cycling can be provided passively. Alternatively, the apparatus can include one or more thermoelectric cooling elements configured to provide cooling for the PCR cycling

1. Apparatus for performing point of care PCR (polymerase chain reaction) testing, the apparatus comprising:
a reaction chamber configured to hold PCR reagents;
a magnetoresistive sensor array disposed within the reaction chamber;
one or more heating elements disposed to provide heat to the reaction chamber;
wherein the magnetoresistive sensor array includes two or more sensor elements;
wherein the two or more sensor elements are used as temperature sensors for the reaction chamber;
a processor configured to provide temperature control of the reaction chamber for PCR cycling by controlling the one or more heating elements responsive to signals from the temperature sensors;
wherein T₀ is a reference temperature, wherein R₀ⱼ is zero field resistivity of sensor element j at temperature T₀, wherein α is a temperature coefficient of the magnetoresistive sensor array, and wherein a zero field resistivity Rⱼ of sensor element j is given by Rⱼ=R₀ⱼ (1+α(T−T₀));
wherein a temperature calibration of the sensor elements is determined by determining α for the magnetoresistive sensor array and determining R₀ⱼ for each sensor element.

2. The apparatus of claim 1:
further comprising a substrate;
wherein the magnetoresistive sensor array is disposed on a first surface of the substrate;
wherein the one or more heating elements are disposed on a second surface of the substrate opposite the first surface of the substrate;
wherein a floor of the PCR reaction chamber is formed by the substrate such that the magnetoresistive sensor array is inside the PCR reaction chamber.

3. The apparatus of claim 2, further comprising one or more thermally conductive vias configured to enhance heat flow from the one or more heating elements to the PCR reaction chamber.

4. The apparatus of claim 3, further comprising a thermally conductive plate sandwiched between the magnetoresistive sensor array and the substrate, wherein the thermally conductive plate is in physical contact with the one or more thermally conductive vias.

5. The apparatus of claim 1, wherein a temperature of the PCR reaction chamber is measured by averaging the temperatures obtained from each sensor element.

6. The apparatus of claim 1, wherein the temperature coefficient of the magnetoresistive sensor array a is determined in a separate calibration step.

7. The apparatus of claim 1, wherein the reference temperature T₀ is room temperature, and wherein determining R₀ⱼ for each sensor element j is done by measuring zero-field resistivity at room temperature for each sensor element.

8. The apparatus of claim 1, wherein the processor is configured to provide end-point PCR detection or wherein the processor is configured to provide real-time PCR detection.

9. The apparatus of claim 8, wherein the PCR reagents include a secondary detection probe configured to bind to magnetic nanoparticles and configured to bind to a target species to be detected.

10. The apparatus of claim 9, wherein the real-time PCR detection cycles between a detection phase and non-detection phases, wherein the secondary detection probe is bound to the target species during the detection phase, and wherein the secondary detection probe is not bound to the target species during the non-detection phases.

11. The apparatus of claim 8, wherein the secondary detection probe is complementary to a common sequence included in the PCR primers for every target species, whereby the secondary detection probe can be used universally for all target species in a multiplexed real-time PCR reaction.

12. The apparatus of claim 1, wherein a current provided to the sensor elements is modulated at a first frequency f₁, wherein a magnetic field provided to the sensor elements is modulated at a second frequency f₂ distinct from the first frequency, and wherein a signal of interest from the sensor elements is at a sum frequency f₁+f₂ or at a difference frequency f₁−f₂.

13. The apparatus of claim 12, further comprising analog filtering circuitry configured to pass the signal of interest while suppressing other signals at frequencies f₁ and f₂.

14. The apparatus of claim 1, wherein the PCR reagents include a DNA polymerase having an antibody-mediated hot start property, whereby the DNA polymerase will not deactivate during hybridization steps of the PCR cycling.

15. The apparatus of claim 1, wherein the PCR reagents have a glycerol content of 0.5% or less by volume, whereby an effect of fluid viscosity on magnetic nanoparticle diffusion is negligible.

16. The apparatus of claim 1, wherein the heating elements are resistive Joule heating elements.

17. The apparatus of claim 1, wherein cooling for the PCR cycling is provided passively.

18. The apparatus of claim 1, further comprising one or more thermoelectric cooling elements configured to provide cooling for the PCR cycling.

19. The apparatus of claim 1, wherein the one or more heating elements are two or more heating elements, and wherein the two or more heating elements are individually controlled in response to temperature signals from the two or more sensor elements to reduce temperature variation of the two or more sensor elements.
